(19) United States
(12) Patent Application Publication
(10) Pub. No.: US 2016/0220762 A1
(43) Pub. Date: Aug. 4, 2016

(54) NEEDLE ASSEMBLY WITH SITE PREPARATION PROVISIONS

(71) Applicant: Smiths Medical ASD, Inc., Rockland, MA (US)

(72) Inventors: David Goral, Brookfield, CT (US); Gursel Akcay, Madison, CT (US); Kathryn Felicito, Cheshire, CT (US); Thomas Koehler, Simsbury, CT (US)

(21) Appl. No.: 15/012,032

(22) Filed: Feb. 1, 2016

Related U.S. Application Data
(60) Provisional application No. 62/109,715, filed on Jan. 30, 2015.
(60) Provisional application No. 62/109,722, filed on Jan. 30, 2015.
(60) Provisional application No. 62/109,742, filed on Jan. 30, 2015.
(60) Provisional application No. 62/109,745, filed on Jan. 30, 2015.
(60) Provisional application No. 62/109,750, filed on Jan. 30, 2015.
(60) Provisional application No. 62/109,755, filed on Jan. 30, 2015.
(60) Provisional application No. 62/109,735, filed on Jan. 30, 2015.
(60) Provisional application No. 62/109,673, filed on Jan. 30, 2015.
(60) Provisional application No. 62/109,710, filed on Jan. 30, 2015.
(60) Provisional application No. 62/109,759, filed on Jan. 30, 2015.
(60) Provisional application No. 62/109,766, filed on Jan. 30, 2015.

(51) Int. Cl. A61M 5/32; A61M 21/02; A61M 5/42

(52) U.S. Cl. CPC A61M 5/3202; A61M 5/422; A61M 21/02; A61M 2021/0016

(57) ABSTRACT

A needle assembly with a self-contained antiseptic swab for treatment of a biological site of a patient. The self-contained antiseptic swab includes a sleeve, an absorbent material containing an antiseptic agent, and operably carried by an exterior surface of the sleeve, and the cap is removably coupled to the sleeve, such that when coupled to the sleeve an interior surface of the cap and the exterior surface of the sleeve form a substantially sealed chamber encasing the absorbent material, preserving the integrity of the antiseptic agent.

### RELATED APPLICATIONS

The present application claims the benefit of U.S. Provisional Application Nos. 62/109,673; 62/109,710; 62/109,715; 62/109,722; 62/109,735; 62/109,742; 62/109,745; 62/109,750; 62/109,755; 62/109,759; 62/109,766, all of which were filed Jan. 30, 2015 and are hereby incorporated herein by reference.

### TECHNICAL FIELD

The present disclosure relates generally to needle assemblies, and more particularly to a needle assembly that includes an antiseptic sheath and/or tourniquet.

### BACKGROUND

Intravenous (IV) therapy is a versatile technique used for the administration of medical fluids to and withdrawal of bodily fluids from patients. IV therapy has been used for various purposes such as the maintenance of fluid and electrolyte balance, the transfusion of blood, the administration of nutritional supplements, chemotherapy, and the administration of drugs and medications. Fluids may be administered intravenously by injection through a hypodermic needle, or intermittently or continuously by infusion using a needle or catheter. The most common intravenous access method utilized by clinicians is the peripheral IV catheter.

A peripheral IV catheter is made of soft, flexible plastic or silicone rubber, generally between fourteen to twenty-four gauge in size. In the conventional venipuncture procedure, a catheter is inserted into a vein in the patient's hand, foot, or the inner aspect of the arm or any vein in the body that will accept an IV catheter. In order to properly place the IV catheter in a patient's vein, a sharp introducer needle must be used to puncture the skin, tissue, and vein wall to provide a path for placement of the catheter in the vein.

Conventional IV needle assemblies include an “over-the needle” catheter where the catheter is coaxially placed over a needle. The catheter thus rides with the needle through the skin, tissue, and vein wall and into the patient's vein. When the needle pierces the vein, blood will “flashback” into the needle. Once a clinician observes this flashback of blood, the clinician will know that the catheter and needle have been inserted in the vein. The catheter can be advanced further into the vein as desired and the needle can then be withdrawn from the catheter.

One such example of this type needle assembly is marketed by Smiths Medical ASD, Inc. of St. Paul, Minn., under the JELCO trademark, as described in U.S. Pat. No. 5,000,740 (depicting an IV catheter insertion device marketed by Smiths Medical ASD, Inc. under the PROTECTIV trademark), U.S. Pat. No. 7,736,342 (depicting an IV catheter insertion device marketed by Smiths Medical ASD, Inc. under the VIAVALVE trademark), and U.S. Pat. Nos. 7,291,130 and 8,257,322 (depicting an IV catheter insertion device marketed by Smiths Medical ASD, Inc. under the INTUITIV Safety IV Catheters trademark), all of which are incorporated by reference herein.

Before a peripheral IV catheter can be employed, the biological site on the patient's skin is normally prepared by a clinician. Typically a tourniquet is applied proximal to the biological site and a variety of techniques are used to dilate the patient's vein. The biological site where the needle and catheter are to be inserted is then disinfected. Otherwise, bacteria on the patient's skin will be able to enter the patient's body and infect the patient. Typically, a cotton swab, either held directly by the clinician or indirectly via an integral handle or a separate handle such as by the use of forceps, is soaked with an antiseptic or antiseptic agent. Such antiseptic agents commonly include alcohol, povidone iodine or chlorhexidine. The cotton swab is rubbed over the patient's skin to thoroughly wet the skin with the antiseptic agent and thus disinfect the patient's skin. After the patient's skin is disinfected, the cotton swab is discarded and the skin is punctured with the needle and catheter. Once the catheter is inserted the tourniquet can be removed.

Although this procedure is generally satisfactory, it could be improved. For example, the medical device and the antiseptic agent are typically supplied separately. Indeed, in many instances the antiseptic agent is also supplied separately from the cotton swab or applicator. This results in separate inventory management for the healthcare entity which can be expensive. In addition, in situations, the clinician may waste valuable time in locating the proper antiseptic agent and applicator before the medical procedure can begin; this can be particularly true in the excited and noisy environment of a trauma scene or ambulance ride. In addition, many traditional applicators require the clinician to directly contact the antiseptic agent during application to the patient. Finally, in many developing countries, the proper disinfecting protocol may not be followed because of the shortcomings described above, lack of training, or other reasons.

The same inventory, ease of use and efficiency problems apply to use of a tourniquet. Moreover, clinicians might reuse a given tourniquet multiple times before disposal, thereby potentially exposing subsequent patients to the risk of exposure of bodily fluids of earlier patients that made contact with the tourniquet.

Accordingly, the applicants have identified a need for a more efficient way of providing an antiseptic agent and tourniquet when preparing a biological site on a patient for insertion of a peripheral IV catheter. In particular, the applicants have identified a need for a way of storing the antiseptic agent and tourniquet with the needle assembly, so that proper protocol is more inherently provided, and so that a clinician can save time and effort in properly preparing a biological site, without unduly changing the design or manufacturing procedures of existing needle assemblies.

### SUMMARY OF THE DISCLOSURE

Embodiments of the present disclosure meet the above described need by providing a self-contained antiseptic swab or tourniquet that can be incorporated into existing needle assembly designs for the purpose of minimizing the number of separate items and steps needed to prepare a biological site of a patient before insertion of a peripheral IV catheter.

One embodiment of the present disclosure provides a needle assembly with a self-contained antiseptic swab assembly for treatment of a biological site of a patient. The needle assembly has an insertion needle, a needle hub, and an antiseptic needle sheath assembly that includes a protective needle sheath, and a self-contained antiseptic swab assembly. The insertion needle has a sharpened distal tip and a proximal end. The needle hub is operably coupled to the proximal end of the insertion needle. The protective needle sheath is operably coupled to the needle hub. The protective needle sheath has a tubular wall for housing the insertion needle. The self-contained antiseptic swab assembly is adapted for the treatment of the biological site of the patient, and includes a sleeve, an absorbent material and a cap. The sleeve is operably coupled to an exterior of the tubular wall of the protective needle sheath. The absorbent material contains an antiseptic agent, and is operably carried by an exterior surface of the sleeve. The cap has a proximal end and a distal end. The proximal end is removably coupled to the sleeve, such that when coupled to the sleeve the interior surface of the cap and the exterior surface of the sleeve form a substantially sealed chamber encasing the absorbent material.

In some versions, the antiseptic agent comprises at least one of ethyl and isopropyl. In some versions, the absorbent material further contains an analgesic agent. In some versions, the material further contains an aromatherapy agent. In some versions, the material further contains a visual indicator configured to visually indicate that the antiseptic agent has been applied at the biological site for a sufficient length of time. In some versions, the visual indicator is at least one of a thermochromatic indicator, a photochromic indicator and a redox indicator. In some versions, the absorbent material is wrapped around the sleeve and is configured to be at least partially unwrapped from the sleeve upon removal of the cap to aid in applying the antiseptic agent to the biological site of the patient. In some versions, the absorbent material comprises a bulbous tip to aid in applying the antiseptic agent to the biological site of the patient. In some versions, the cap includes a line of perforations configured to weaken a portion of the cap to aid in opening the substantially sealed chamber for exposure of the absorbent material. In some versions, the cap is coupled to the sleeve by one or more living hinges. In some versions, the self-contained antiseptic swab assembly further includes a tourniquet. In some versions, the tourniquet is wrapped around the cap, and is configured to be unwrapped from the cap to aid preparing the biological site of the patient for insertion of the needle.

Another embodiment of the present disclosure provides a self-contained antiseptic swab assembly for treatment of a biological site of a patient that can be coupled to an existing protective needle sheath of a needle assembly. The self-contained antiseptic swab assembly includes a sleeve, an absorbent material, and a cap. The sleeve is operably couplable to a protective needle sheath of a needle assembly. The absorbent material contains an antiseptic agent, and is operably carried by an exterior surface of the sleeve. The cap has a proximal end and a distal end, wherein the proximal end is removably coupled to the sleeve, such that when coupled to the sleeve the interior surface of the cap and the exterior surface of the sleeve form a substantially sealed chamber encasing the absorbent material.

In some versions, the antiseptic agent comprises at least one of ethyl and isopropyl. In some versions, the absorbent material further contains an analgesic agent. In some versions, the material further contains an aromatherapy agent. In some versions, the material further contains a visual indicator configured to visually indicate that the antiseptic agent has been applied at the biological site for a sufficient length of time. In some versions, the visual indicator is at least one of a thermochromatic indicator, a photochromic indicator and a redox indicator. In some versions, the absorbent material is wrapped around the sleeve, and is configured to be at least partially unwrapped from the sleeve upon removal of cap to aid in applying the antiseptic agent to the biological site of the patient. In some versions, the absorbent material comprises a bulbous tip to aid in applying the antiseptic agent to the biological site of the patient. In some versions, the cap includes a line of perforations configured to weaken a portion of the cap to aid in opening the substantially sealed chamber for exposure of the absorbent material. In some versions, the cap is coupled to the sleeve by one or more living hinges. In some versions, the self-contained antiseptic swab assembly further includes a tourniquet. In some versions, the tourniquet is wrapped around the cap, and is configured to be unwrapped from the cap to aid preparing the biological site of the patient for insertion of the needle.

The summary above is not intended to describe each illustrated version or every implementation of the present disclosure. The figures and the detailed description that follow more particularly exemplify these versions.

### BRIEF DESCRIPTION OF THE DRAWINGS

The disclosure can be more completely understood in consideration of the following detailed description of various versions of the disclosure, in connection with the accompanying drawings, in which:

FIG. 1 is a perspective, top view of a peripheral intravenous catheter of the prior art, including a protective needle sheath in a first position.

FIG. 2 is a perspective, top view of the peripheral intravenous catheter of FIG. 1 in a second position.

FIG. 3 is a perspective, bottom view of the peripheral intravenous catheter of FIG. 1 in a first position.

FIG. 4 is a perspective, bottom view of the peripheral intravenous catheter of FIG. 1 in a second position.

FIG. 5 is an exploded, perspective view depicting protective needle sheath 166 and self-contained antiseptic swab assembly of a first embodiment of an antiseptic needle sheath in accordance with the disclosure.

FIG. 6 is a perspective view depicting a first embodiment of a needle assembly in accordance with the disclosure.

FIG. 7 is a perspective view depicting the needle assembly of FIG. 6 in a second configuration.

FIG. 8 is a perspective view depicting a second embodiment of a needle assembly in accordance with the disclosure.

FIG. 9 is a perspective view depicting the needle assembly of FIG. 8 in a second configuration.

FIG. 10 is a perspective view depicting a third embodiment of a needle assembly in accordance with the disclosure.

FIG. 11 is a perspective view depicting the needle assembly of FIG. 10 in a second configuration.

FIG. 12 is a perspective view depicting a fourth embodiment of a needle assembly in accordance with the disclosure.

FIG. 13 is a perspective view depicting a fifth embodiment of a needle assembly in accordance with the disclosure.

FIG. 14 is perspective view depicting a sixth embodiment of a needle assembly in accordance with the disclosure.

FIG. 15 is a perspective view depicting the needle assembly of FIG. 14 in a second configuration.

FIG. 16 is a perspective view depicting a seventh embodiment of a needle assembly in accordance with the disclosure.

While embodiments of the disclosure are amenable to various modifications and alternative forms, specifics thereof shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure as defined by the appended claims.

### DETAILED DESCRIPTION

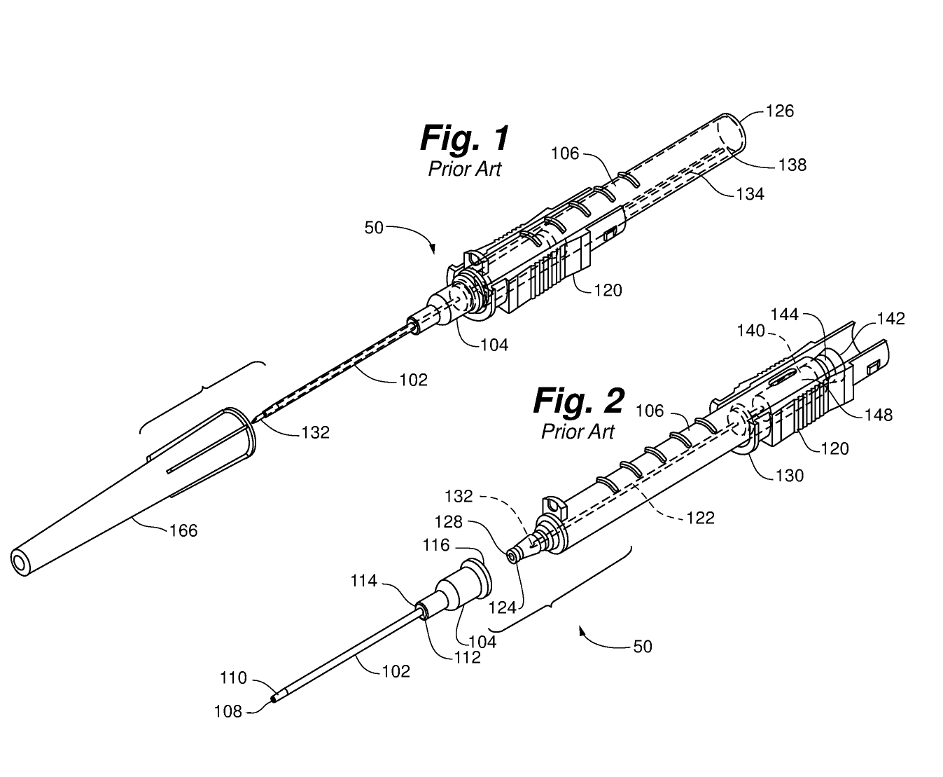

Referring to FIGS. 1-4, an example of a conventional needle assembly 50, as described in the Background section is depicted. Conventional needle assemblies for use in inserting a peripheral IV catheter generally include a catheter 102, a catheter hub 104, an insertion needle mechanism 106, and a protective needle sheath 166.

Catheter 102 is made of soft, flexible plastic or silicone rubber and is hollow, thereby defining a lumen 108 that runs substantially parallel to the longitudinal axis of the catheter 102. Catheter 102 is further defined by a distal end 110 and a proximal end 112. Distal end 110 is configured for insertion into a biological site of a patient, such as a vein. As such, the distal end 110 can be tapered in an effort to minimize the amount of force required to insert the catheter 102 into the biological site. Distal end 110 of catheter 102 can also be relatively blunt, so that the catheter 102 alone is unable to pierce the skin of a patient or clinicians, thereby reducing the risk of an inadvertent needle stick.

Proximal end 112 of the catheter 102 can be fixedly coupled to the catheter hub 104. In some versions, the catheter hub 104 is configured to control the flow of fluid through the catheter lumen 108 and has a distal end 114, a proximal end 116, and an internal fluid passageway (not depicted) that runs substantially parallel to the longitudinal axis of the catheter hub 104 between the distal end 114 and the proximal end 116.

In some versions, the fluid passageway includes a septum or valve to enable sealing of the fluid passageway to restrict or prevent bodily fluid from leaking out of the catheter hub 104 when the catheter 102 is inserted into a patient's vein and the insertion needle 122 is removed from the catheter hub 104. The valve can be configured in such a way that at times fluid is free to flow through the catheter hub 104 and catheter lumen 108, and at other times the flow of fluid through the catheter hub 104 and catheter lumen 108 is restricted or stopped.

In particular, the valve can be in an open position, for example, when the insertion needle 122 passes through the catheter hub 104, thereby enabling fluid to flow from the patient's vein into a flash chamber 140. The valve can be in a closed position, for example, when the insertion needle 122 is withdrawn from the catheter hub, to restrict or prevent fluid from leaking out of the catheter hub 104 when the insertion needle 122 is removed. In some versions, the valve can be opened and closed by applying a compressive force to a portion of the valve or the septum. For example, the valve can be opened when a luer lock or other needleless connector is coupled to the catheter 104 and applies a compressive force against valve. Conversely, the valve can be closed when the needleless connector is disconnected, thereby removing the compressive force against the valve. In one version, the passageway is biased closed. In this manner, valve or septum selectively controls the flow of fluid through catheter lumen 108. Various catheter hub designs having a septum and/or a valve are disclosed in a concurrently filed application entitled “Intravenous Catheter Assembly Design,” Attorney Docket No. 4176.191US02, which is incorporated by reference herein.

Insertion needle mechanism 106 generally includes a needle housing 118 and a needle hub 120 coupled to the needle 122. Needle housing 118 has a distal end 124 and a proximal end 126. The distal end 124 of the needle housing 118 can be configured with a tapered blunt tip sized to create a friction fit with a portion of catheter hub 104. In some versions, distal end 124 can be at least partially inserted into a socket defined in catheter hub 104, such that distal end 124 applies a compressive force to a portion of catheter hub 104, thereby opening the internal fluid passageway of catheter hub 104. Distal end 124 can further define an aperture 128 through 10 which insertion needle 122 can pass. In some embodiments, the needle housing 118 can include a catheter hub coupling and release mechanism configured to selectively couple to and release from catheter hub 104. Various catheter hub coupling and release mechanism are disclosed in a concurrently filed application entitled “Releaseable Catheter Hub Retainer,” Attorney Docket No. 4176.180US02, which is incorporated by reference herein.

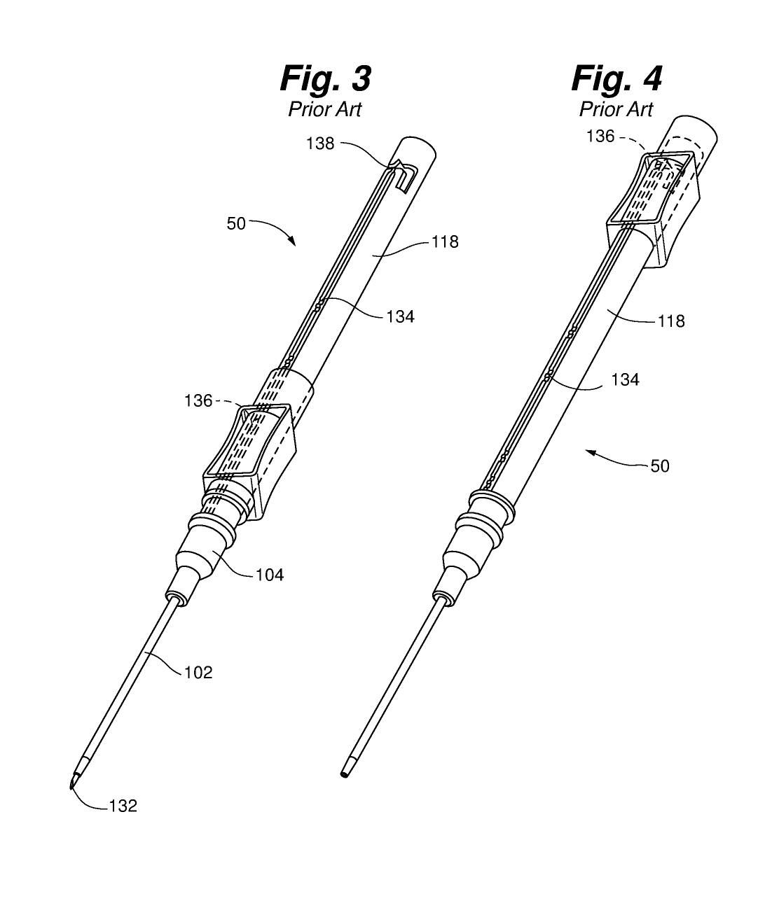

Needle hub 120 can be operably coupled to the insertion needle 122, can be slideably coupled to the needle housing 118. For example, the needle hub 120 can have a “C” shaped cross section 130 conformed to fit around the outer surface of the needle housing 118 in a manner that prevents the needle hub 120 from readily separating from the needle housing 118, yet enables the needle hub 120 to slide along the longitudinal axis of the needle housing 118 with minimal resistance. In some versions, the needle hub 120 can be configured to slide along a groove 134 defined in needle housing 118 to restrict the needle hub 120 from rotating about the longitudinal axis of the needle housing 118. Needle hub 120 can include a protuberance 136 (as depicted in FIGS. 3-4) configured to fit within the groove 134 of the needle housing 118, thereby enabling linear movement of the needle hub 120 substantially parallel to the longitudinal axis of the needle housing 118, but restricting rotational movement of the needle hub 120 relative to the needle housing 118.

In some versions, the needle hub 120 is slideable between a first position (as depicted in FIGS. 1 and 3) and a second position (as depicted in FIGS. 2 and 4). In the first position, a portion of the insertion needle 122 extends through the needle guard aperture 128, the catheter hub 104 and the catheter lumen 108, such that the sharpened tip 132 protrudes slightly beyond the distal end 110 of the catheter 102. In the second position, the insertion needle 122 is withdrawn from the catheter lumen 108 and the catheter hub 104 and the sharpened tip 132 is shielded by the needle housing 118 in a manner intended to reduce or eliminate the likelihood of an inadvertent needle stick.

Insertion needle 122 can be locked in position relative to the needle housing 118. Several different types of locking mechanisms can be used for this purpose. For example, the groove 134 of needle housing 118 can have a bottleneck 138 defined in it, where the bottleneck 138 portion of groove 134 generally has a narrower width than the rest of groove 134. Protuberance 136 of the needle hub 120 can be triangular or wedge-like in shape (as depicted in FIGS. 3 and 4) where the apex of the wedge faces the bottleneck 138 when in the first position. When an external force is applied to the needle hub 120 in an effort to slide it into the second position, the apex of the wedge of protuberance 136 will come into contact with bottleneck 138. Bottleneck 138, which can have a width narrower than that of the protuberance 136 will initially resist movement of the protuberance 136 through bottleneck 138. However, with sufficient force the wedge-shape protuberance 136 will cause the bottleneck 138 to temporarily deform, thereby enabling the protuberance 136 to pass through the bottleneck 138. Thereafter the protuberance 136 will be unable to pass back through the bottleneck 138 in the opposite direction, and the insertion needle 122 will be locked in position relative to needle housing 118.

Needle hub 120 further includes a flash chamber 140. Flash chamber 140 can be configured as a cavity in fluid communication with the lumen of the insertion needle 122 opposite sharpened tip 132. Flash chamber 140 can be constructed of a transparent or translucent material to enable a clinician to visually see when fluid enters the flash chamber 140. The proximal end 142 of flash chamber 140 can be plugged with a microporous flash plug 144. Flash plug 144 can be comprised of a material that enables air to vent from the flash chamber 140 as fluid fills the chamber, but inhibit the fluid from passing from the flash chamber 140. In some embodiments, flash chamber 140 can further include a diagnostic sampling port configured to enable selective access to fluid contained within flash chamber 140. Various needle assemblies having diagnostic sampling ports are disclosed in a concurrently filed application entitled “Needle Assembly with Diagnostic Analysis Provisions,” Attorney Docket No. 4176.178US02, which is incorporated by reference herein.

Protective needle sheath or cover 166 can be selectively coupled to the distal end 124 of the needle housing 118 and/or the needle hub 120 for the purpose of safely housing the insertion needle 122 when the insertion needle 122 is exposed and has the potential to cause an inadvertent needle stick, particularly when convention the needle assembly 50 is in the first position. Protective needle sheath 166 can be coupled to the distal end 124 by a friction fit, or by a simple locking mechanism.

Versions of the present disclosure enable a modular self-contained antiseptic swab assembly or tourniquet to be affixed to the protective needle sheath 166 of a conventional insertion needle mechanism 106.

### A. First Embodiment

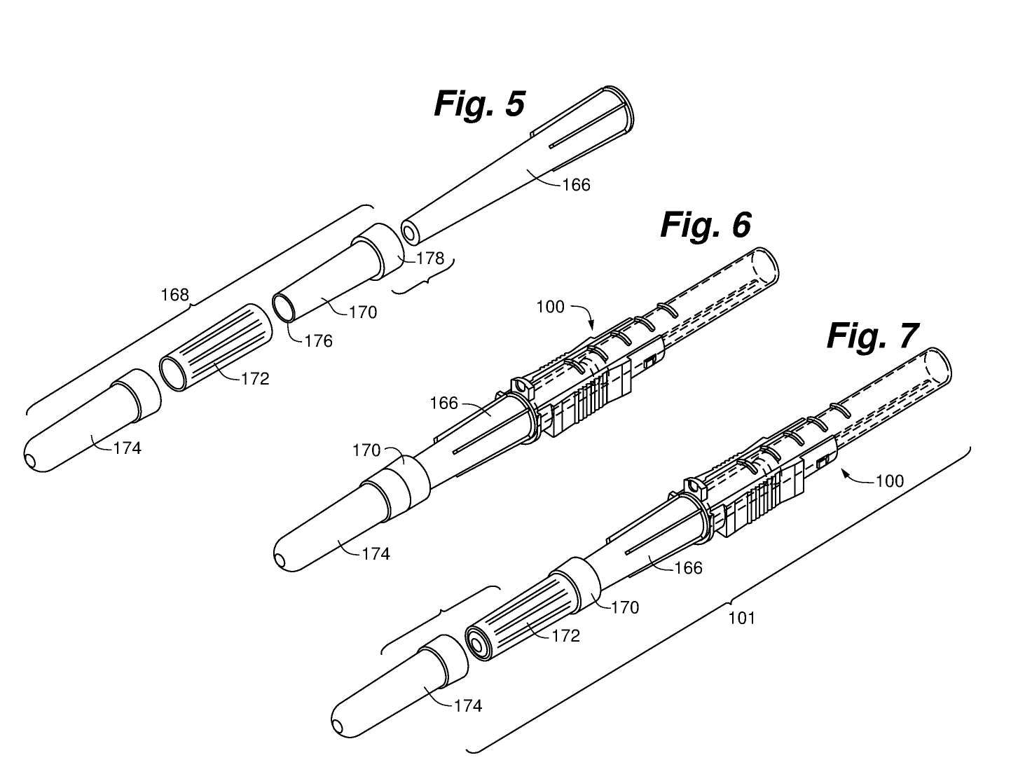

Referring to FIGS. 5-7, a needle assembly 100 according to a first embodiment of the disclosure is depicted. The needle assembly 100 has a self-contained antiseptic swab assembly 168. In one version, self-contained antiseptic swab assembly 168 is retrofit, so that the design and manufacture of the protective needle sheath 166 and other components of the needle assembly 100 do not need to be altered.

In one version, the self-contained antiseptic swab assembly 168 includes a sleeve 170, absorbent material 172 and a cap 174. In one version, the sleeve 170 is a thin walled tubular structure defined by a first end 176 and a second end 178. Sleeve 170 can be shaped and sized to create a friction fit with the protective needle sheath 166, such that the sleeve 170 is secured in a fixed position when placed over a portion of protective needle sheath 166, with the self-contained antiseptic swab assembly 168 and protective needle sheath 166 making up an antiseptic needle sheath assembly 101. In other versions, sleeve 170 can be mechanically affixed, glued, or sonically welded to protective needle sheath 166.

Absorbent material 172 can be fixedly coupled to the sleeve 170. Absorbent material 172 can be constructed from foam, woven, non-woven, or other type of absorbent material. Absorbent material 172 can be, for example, constructed of cotton, nylon, rayon, urethane or a combination thereof. In one version, absorbent material 172 is configured to be tubular in shape and is shaped and sized to fit over the exterior surface of sleeve 170. Absorbent material 172 can be fixedly coupled to sleeve 170 by friction fit, threaded engagement, solvent bonding, sonic welding, by use of an adhesive, or a combination thereof. Absorbent material 172 can be soaked in an antiseptic agent, antimicrobial agent, germicideal agent, antibacterial agent, or another agent for the purpose of reducing the chance of infection or inflammation of the biological site of the patient. For example, in one version, the absorbent material 172 can be soaked in ethyl or isopropyl alcohol. In other versions, the absorbent material 172 can be soaked in a drug or medicament for intended delivery to the patient. In still other versions, the absorbent material 172 can be soaked in an analgesic agent for pain relief, and/or an aromatherapy agent to provide a pleasing or medicinal aroma.

Cap 174 can be selectively coupled to sleeve 170 for the purpose of providing a substantially sealed chamber between the sleeve 170 and the cap 174, thereby encasing the absorbent material 172. Thus, in some versions, the cap 174 surrounds and provides an air and fluid tight cover to protect the integrity of the absorbent material 172, so that the agent contained therein does not dry out or lose its chemical potency. In some versions, the cap 174 can be a rigid tubular member that is removed from the sleeve 170 much like the cap of a pen. In other versions, the cap 174 can be a flexible membrane or shrink wrap that is torn off or peeled from the sleeve 170 in order to expose the absorbent material 172.

### B. Second Embodiment

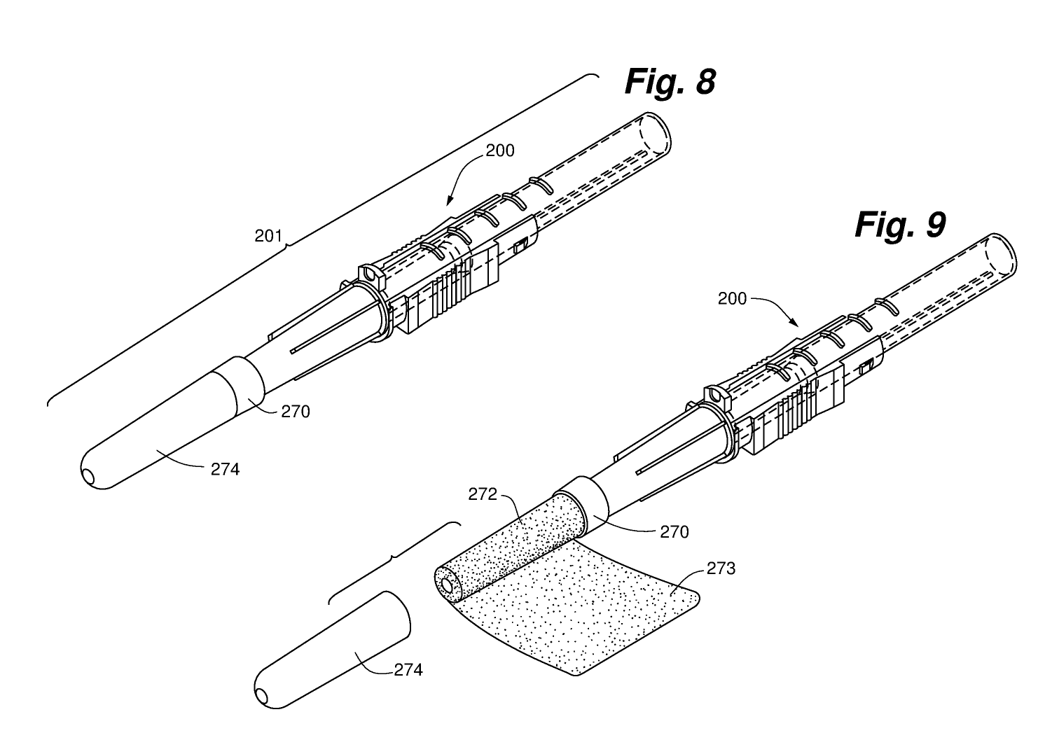

Referring to FIGS. 8 and 9, a needle assembly 200 according to a second version of the disclosure that includes an antiseptic needle sheath assembly 201 is depicted. In this embodiment, the absorbent material 272 can be affixed to the sleeve 270 such that an appendage portion 273 or flap of the absorbent material 272 has a larger degree of movement relative to the sleeve 270. In this version, the appendage portion 273 can be rolled around the remainder of the absorbent material 272 and the first end 276, when the cap 274 is coupled to the second end 278. When the cap 274 is removed, the appendage portion 273 can be unrolled and used in a manner that enables improved contact with the biological site of the patient. In other versions, the absorbent material 272 is completely removable from the sleeve 270 prior to use. In some versions, a sealed pack containing the absorbent material 272 can be removably coupled to the needle assembly 200.

### C. Third Embodiment

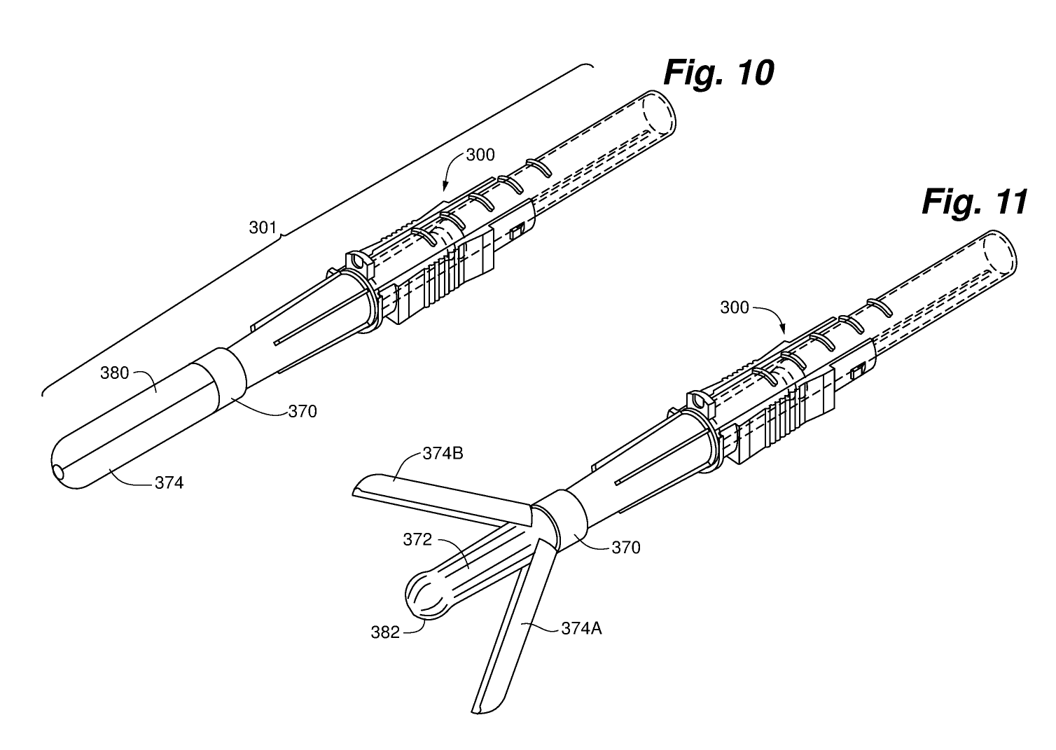

Referring to FIGS. 10 and 11, a needle assembly 300 according to a third embodiment of the disclosure that includes an antiseptic needle sheath assembly 301 is depicted. In this embodiment, the absorbent material 372 can include a rounded, blunted or bulbous tip 382 configured to further enable improved contact with the biological site of the patient. In one version, the cap 374 can be comprised of two or more parts 374A, 374B that separate to provide access to the absorbent material 372. The two or more cap parts 374A, 374B can be held to each other by a weakened or perforated seam that can be separated or broken when access to the absorbent material 372 is desired. In one version, the two or more second cap parts 374A, 374B can be coupled to one another by a living hinge, so that after use self-contained antiseptic swab assembly can be disposed of in a single piece.

### D. Fourth Embodiment

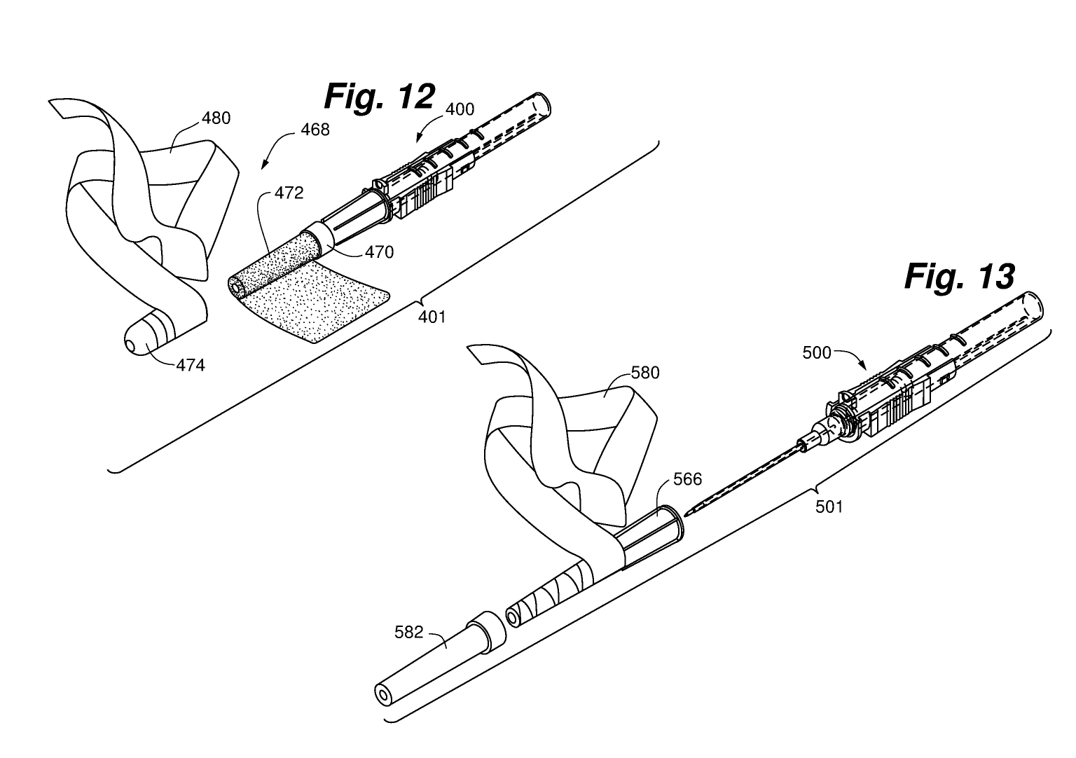

Referring to FIG. 12, a needle assembly 400 according to a fourth embodiment of the disclosure that includes an antiseptic needle sheath assembly 401 is depicted. In this embodiment, the self-contained antiseptic swab assembly 468 includes sleeve 470, absorbent material 472, cap 474, and tourniquet 480. Tourniquet 480 can be rolled, wound, wrapped around, or otherwise removably coupled to an end cap or cap 474. In some versions, tourniquet 480 is coupled to cap 474 by an adhesive. When cap 474 is removed, tourniquet 480 can be unrolled and used in a preparation of the biological site of the patient. Tourniquet 480 can be designed for single-use. In some versions, tourniquet 480 can be resilient or elastic. In one version, tourniquet 480 can be constructed of a flat rubber material.

### E. Fifth Embodiment

Referring to FIG. 13, a needle assembly 500 according to a fifth embodiment of the disclosure that includes an antiseptic needle sheath assembly 501 is depicted. In this embodiment, the tourniquet 580 can be removably coupled to protective needle sheath 166. In this version, a tourniquet cover 582 can be selectively coupled to the protective needle sheath 566 for the purpose of providing a substantially sealed chamber between the protective needle sheath 566 and the tourniquet cover 582 encasing the tourniquet 580. In some versions, the tourniquet cover 582 can be a rigid tubular member that is removed from the protective needle sheath 566 much like the cap of a pen. In other versions, the tourniquet cover 582 can be a flexible membrane or shrink wrap that is torn off or peeled from the protective needle sheath 566 in order to expose the tourniquet 580.

### F. Sixth Embodiment

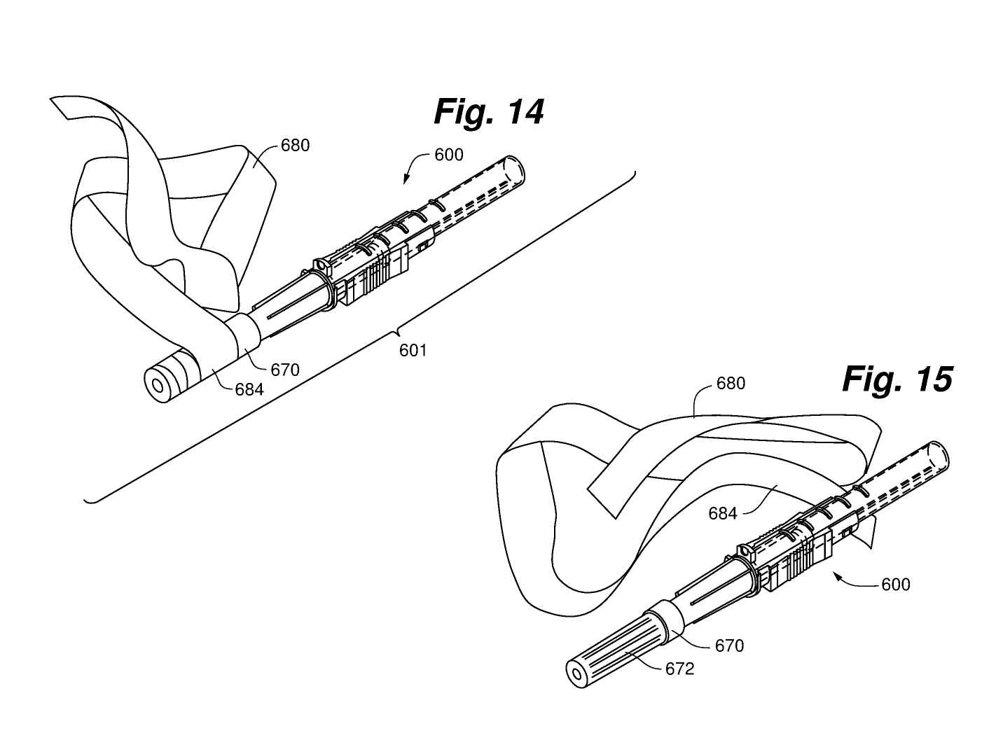

Referring to FIGS. 14 and 15, a needle assembly 600 according to a sixth embodiment of the disclosure that includes an antiseptic needle sheath assembly 601 is depicted. In this embodiment, the tourniquet 680 substantially surrounds the absorbent material 672 to serve as an end cap or cover for the absorbent material 672. In this version, the tourniquet 680 can be rolled, wound, wrapped around, or otherwise removably coupled to the absorbent material 672, such that removal of the tourniquet 680 also removes a protective covering 684 or coating over the absorbent material 672, thereby exposing the absorbent material 672 to the air. A cap or tourniquet cover can be used in combination with this embodiment. In the alternative, the tourniquet 680 can be coupled directly to the sleeve 670.

### G. Seventh Embodiment

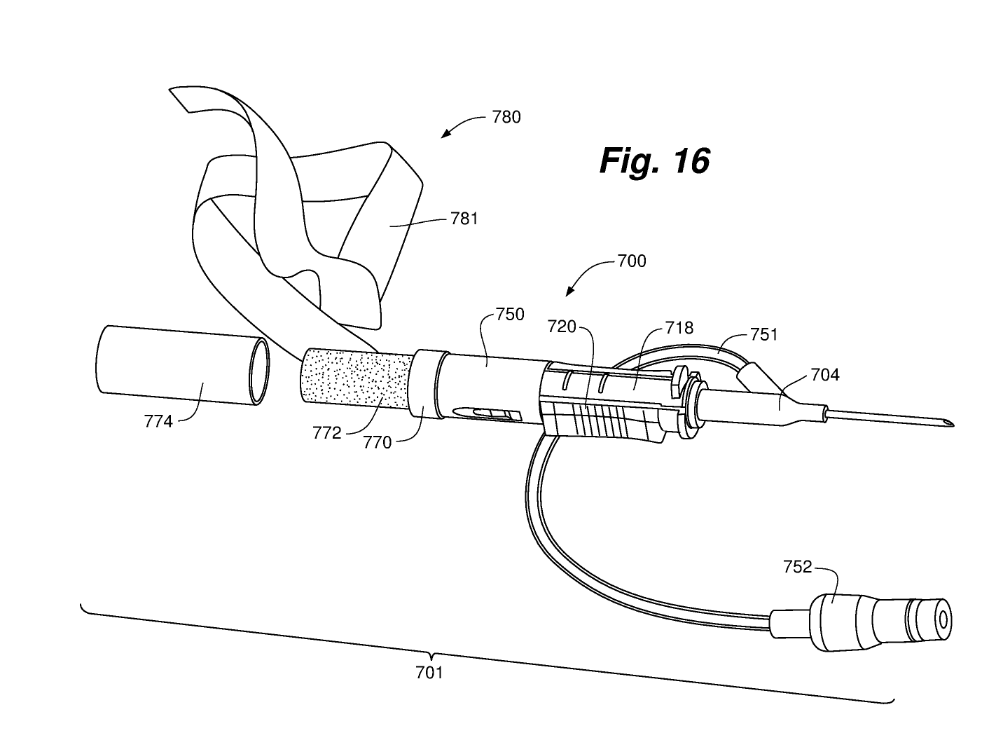

Referring to FIG. 16, a needle assembly 700 according to a seventh embodiment of the disclosure is depicted. In this embodiment, the needle assembly 700 further includes an end cap 780 configured to cover a portion of the needle housing 718 proximate to the proximal end to reduce the likelihood of unwanted or uncontrolled movement of the needle housing 718 relative to the needle hub 720 by the user or clinician's palm or inside of their hand during catheter insertion.

In some versions, the end cap 780 can include a self-contained antiseptic swab assembly including sleeve 770, absorbent material 772, and cap 774, as well as a tourniquet 781. In some versions, the catheter hub 704 can be operably coupled to hollow tubing 751 and a tube connector 752.

### H. Universal Time Indicator

In some versions, the antiseptic wipe can include a time indicator, such as a visual indicator, for indicating that the antiseptic has been applied at the insertion site for a sufficient length of time to render antiseptic properties to the insertion site, i.e. adequate disinfection of the site. Preferably, the visual indicator undergoes a color change.

In one particular version, the indicator comprises a thermochromatic indicator, such as a reversible or irreversible thermochromatic ink, dye or pigment. The thermochromatic indicator can be incorporated into the wipe or antiseptic solution, such that it presents a first color when at a first temperature when present on the antiseptic wipe, and then warms to a second temperature greater than the first temperature based on the increased temperature of the patient's skin, causing the indicator to change color upon application of the antiseptic. The indicator then cools due to convection cooling and/or evaporation of the antiseptic, which causes the indicator to change back to its original color or similar. The thermochromatic indicator can be chosen such that the temperature change to shift the indicator takes place over about one minute from application to the patient's skin. Common thermochromatic indicators include liquid crystals such as, for example, cholesteryl nonanoate or cyanobiphenyls, and leuco dyes.

In another version, the color change indicator comprises a photochromic indicator, which is based on the reversible transformation of a chemical species between two forms by the absorption of electromagnetic radiation, such as UV light, where the two forms have different absorption spectra. Some examples of photochromatic indicators include, for example, nitrospiropyran, complexes including stable chromophores such as azobenzene, diarylethene, spiropyran, triarylmethanes, stilbenes, azastilbenes, nitrones, fulgides, spiropyrans, naphthopyrans, spiro-oxazines, quinines, and others.

In this version, the photochromic indicator can be encapsulated, such as in the form of microbeads, that rupture or dissolve upon application of the antiseptic to the patient's skin, such as by rubbing the wipe along the skin. Upon exposure of the photochromic indicator over a length of time, such as one minute, the photochromic indicator changes color, indicating that the insertion site is sufficiently disinfected.

In an alternative version, the cover of the device is formed of a UV barrier, such as a black colored cover. Upon removing the wipe from the cover, the photochromic indicator is exposed to UV light. In this version, the indicator is chosen such that it fires after a slightly longer length of time, likely greater than one minute, to take into account that the wipe may not be used immediately upon removal from the cover.

In yet another version, the color change indicator comprises a redox indicator which undergoes a color change (for example, a color to another color, colorless to color, or color to colorless) at a specific electrode potential, which can be induced upon exposure to air and/or moisture from the air and/or the patient's skin. Such materials can include, for example, pH independent redox indicators, such as, for example, 2,2′-bipyridine (Ru complex), Nitrophenanthroline (Fe complex), N-Phenylanthranilic acid, 1,10-Phenanthroline iron(II) sulfate complex (Ferroin), N-Ethoxychrysoidine, 2,2′-Bipyridine (Fe complex), 5,6-Dimethylphenanthroline (Fe complex), o-Dianisidine, Sodium diphenylamine sulfonate, Diphenylbenzidine, Diphenylamine, Viologen, and pH dependent redox indicators such as, Sodium 2,6-Dibromophenol-indophenol, Sodium 2,6-Dichlorophenol-indophenol, Sodium o-Cresol indophenol, Thionine (syn. Lauth's violet), Methylene blue, Indigotetrasulfonic acid, Indigotrisulfonic acid, Indigo carmine (syn. Indigodisulfonic acid, Indigomono sulfonic acid, Phenosafranin, Safranin T, Neutral red).

Again, in this version, the color change indicator may be encapsulated in a water-soluble microbead or can be incorporated unencapsulated into the antiseptic of the wipe itself. Upon exposure to moisture in the air and/or moisture from the patient's skin, the microbead dissolves or ruptured leaving the exposed color change indicator on the patient's skin. Upon sufficient oxidation or reduction, the color change indicator changes color, indicating that the insertion site is sufficiently cleaned.

As an alternative to all of the versions above, it can be contemplated that the color indicator is incorporated into the cover itself, rather than in the antiseptic wipe. Upon opening of the cover, it triggers the method of color change of the indicator, tailored to the length of time the antiseptic should be left on the insertion site for adequate disinfection. For example, the wipe may first contact the indicator upon removal of the wipe of the cover. Alternatively, removal of the cover first exposes the indicator to electromagnetic radiation, moisture, oxidation, and/or temperature change causing the color change after a predetermined length of time of exposure.

Other time indicators can also be contemplated, such as electronic time indicators printed or otherwise applied to the cover, and which are activated, such as by a membrane switch, upon removal of the cover. Alternatively, an odor indicator can be used which, upon exposure of the indicator to the change in environment from within the cap to the surrounding environment as discussed above with respect to the visual indicators, the indicator undergoes a shift from odorless to aromatic, or vice versa, after a predetermined length of time of exposure.

### I. Operation

In operation, placement of the needle assembly generally includes preparation of the biological site of the patient. In some versions, the tourniquet is removed from the cap, the protective needle sheath, or the absorbent material in accordance with the above described embodiments. The tourniquet 180 is then applied to the patient proximal to the biological site. A variety of techniques are used to dilate the patient's vein.

The clinician cleanses the biological site in accordance with the above described embodiments. In particular, the clinician first removes the cap, thereby exposing absorbent material. The clinician then uses at least a portion of the needle assembly as a handle to apply the agent soaked on the absorbent material to cleanse or treat the biological site. The protective needle sheath, which is coupled to the self-contained antiseptic swab assembly is removed from the needle assembly and disposed of.

After cleansing, the vein is retracted or anchored by placing a thumb over the vein about 50 mm to 75 mm distal to the site. The insertion needle and catheter are introduced into the vein by inserting the bevel of the sharpened tip into the vein at about a twenty to thirty degree angle with the bevel facing up in order to pierce one wall of the vein. If successful, blood from the vein will flow through the lumen of the insertion needle and into the flashback chamber, thereby indicating that the vein has been entered. The needle assembly is lowered towards the skin to decrease the entry angle, and the catheter is advanced slightly into the vein. The insertion needle is loosened and the catheter is gently advanced farther up into the vein until the catheter hub of the catheter is against the biological site. The tourniquet is loosened and the insertion needle is withdrawn from the catheter, as the moveable element is moved from the first position to the second position, so that insertion needle is safely locked within needle housing. Infusion tubing is secured to the catheter hub of the catheter. The catheter is secured to the biological site by gauze and adhesive tape.

Persons of ordinary skill in the relevant arts will recognize that versions may comprise fewer features than illustrated in any individual version described above. The versions described herein are not meant to be an exhaustive presentation of the ways in which the various features may be combined. Accordingly, the versions are not mutually exclusive combinations of features; rather, versions can comprise a combination of different individual features selected from different individual versions, as understood by persons of ordinary skill in the art. Moreover, elements described with respect to one version can be implemented in other versions even when not described in such versions unless otherwise noted. Although a dependent claim may refer in the claims to a specific combination with one or more other claims, other versions can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of Section 112, sixth paragraph of 35 U.S.C. are not to be invoked unless the specific terms “means for” or “step for” are recited in a claim.

What is claimed is:

1. A needle assembly including an antiseptic needle sheath assembly for treatment of a biological site of a patient, comprising:
an insertion needle having a sharpened distal tip and a proximal end;
a needle hub operably coupled to the proximal end of the insertion needle;
a protective needle sheath operably coupled to the needle hub, the protective needle sheath having a tubular wall for housing the insertion needle; and
a self-contained antiseptic swab assembly for the treatment of the biological site of the patient, the self-contained antiseptic swab assembly including—
a sleeve operably coupled to an exterior of the tubular wall of the protective needle sheath,
an absorbent material containing an antiseptic agent, the absorbent material operably carried by an exterior surface of the sleeve, and
a cap having a proximal end and a distal end, the proximal end removably coupled to the sleeve, such that when coupled to the sleeve the interior surface of the cap and the exterior surface of the sleeve form a substantially sealed chamber encasing the absorbent material.

2. The needle assembly of claim 1, wherein the antiseptic agent comprises at least one of ethyl and isopropyl.

3. The needle assembly of claim 1, wherein the absorbent material further contains an analgesic agent.

4. The needle assembly of claim 1, wherein the material further contains an aromatherapy agent.

5. The needle assembly of claim 1, wherein the material further contains a visual indicator configured to visually indicate that the antiseptic agent has been applied at the biological site for a sufficient length of time.

6. The needle assembly of claim 5, wherein the visual indicator is at least one of a thermochromatic indicator, a photochromic indicator and a redox indicator.

7. The needle assembly of claim 1, wherein the absorbent material is wrapped around the sleeve and is configured to be at least partially unwrapped from the sleeve upon removal of the cap to aid in applying the antiseptic agent to the biological site of the patient.

8. The needle assembly of claim 1, wherein the absorbent material comprises a bulbous tip to aid in applying the antiseptic agent to the biological site of the patient.

9. The needle assembly of claim 1, wherein the cap includes a line of perforations configured to weaken a portion of the cap to aid in opening the substantially sealed chamber for exposure of the absorbent material.

10. The needle assembly of claim 1, wherein the cap is coupled to the sleeve by one or more living hinges.

11. The needle assembly of claim 1, wherein the self-contained antiseptic swab assembly further includes a tourniquet.

12. The needle assembly of claim 1, wherein the tourniquet is wrapped around the cap, and is configured to be unwrapped from the cap to aid preparing the biological site of the patient for insertion of the needle.
